(19) United States
(12) Patent Application Publication
(10) Pub. No.: US 2014/0261398 A1
(43) Pub. Date: Sep. 18, 2014

(54) COMBINED MEDICATION ADMINISTRATION AND TRAINING MASK

(71) Applicant: Medical Australia Limited, North Sydney (AU)

(72) Inventors: Morgan David James, Toongabbie (AU); John Fitzpatrick, Scotland Island (AU); Peter Neil Austen, Kellyville (AU); Garry Andrews, Brighton-le-sands (AU)

(73) Assignee: Medical Australia Limited, North Sydney (AU)

(21) Appl. No.: 14/210,714

(22) Filed: Mar. 14, 2014

Related U.S. Application Data
(60) Provisional application No. 61/787,167, filed on Mar. 15, 2013.

(51) Int. Cl. A61D 7/04; A61M 16/14; A61M 16/20; A61M 11/06; A61M 11/00; A61M 16/06; A61M 15/00

(52) U.S. Cl. CPC A61D 7/04; A61M 16/06; A61M 16/14; A61M 15/0085; A61M 11/06; A61M 11/005; A61M 16/20
USPC 128/200.16; 128/203.29; 128/200.14

(57) ABSTRACT

A combined medication and training mask for an animal includes a body, a mask module and a plenum module. A cavity is provided in a terminal end of the mask module. A nebulizer is received in that cavity.

This utility patent application claims the benefit of priority in U.S. Provisional Patent Application Ser. No. 61/787,167 filed on Mar. 15, 2013, the entirety of the disclosure of which is incorporated herein by reference.

### TECHNICAL FIELD

This document relates generally to the field of animal husbandry and more particularly to a combined medication administration and training mask.

### BACKGROUND

The concept of providing an inhalation mask for animals such as horses is generally known in the art. Examples of such masks are shown in, for example, U.S. Pat. No. 5,954,049 to Foley et al. and Published U.S. Patent Application Nos. 2002/0056456 to Foley et al. and 2012/02031252 to Moran. While these masks are useful for their intended purpose they suffer from a number of drawbacks.

This document relates to a multi-purpose or combined medication administration and training mask for animals such as horses, camels and the like. The mask is ergonomically designed and is easy to present, fit and secure in position. The mask is low weight and provides an excellent seal for multiple prolonged therapies or training sessions.

Further, the mask includes an integrated plenum and nebulizer of compact size and reduced weight. The robust design of the mask ensures all critical components are protected from damage and incorporates snap in and snap out delivery features for treatment and training.

Further, the mask is designed for easy cleaning and disinfection. It is then easy to reassemble: the parts click into place and cannot be assembled incorrectly. The mask is also designed to optimize therapy throughout the breathing cycle by (a) minimizing carbon dioxide re-breathing for maximum comfort by fitting closely (low dead-space), (b) ensuring vapor inhalation is prioritized and any excess vapor is flushed through the system with air from the inlet valves and (c) ensuring rapid flow through the inlet, dividing and outlet vents via the largest venting system of any mask currently available.

### SUMMARY

A combined medication administration and training mask for an animal comprises a body having a sidewall, a mask module at a proximal end and a plenum module at a distal end. The mask module includes an open end, a compartment for receiving a muzzle of an animal and a closed end opposite the open end. The plenum module includes a connection end, a closed terminal end and an aerosolizing plenum defined between the connection end and the terminal end. The connection end of the plenum module attaches to the closed end of the mask module. Further the mask includes a cavity in the terminal end of the mask module.

In one possible embodiment a nebulizer is received in the cavity. The nebulizer is held in the cavity and substantially protected from the animal's view and contact by a periphery defined by the sidewall of the body.

In another possible embodiment the mask includes an ultrasonic nebulizer and a jet nebulizer. The ultrasonic nebulizer and jet nebulizer are both sized and shaped to be held in the cavity so that one of the ultrasonic nebulizer and jet nebulizer is held in the cavity of the plenum module at any one time.

The mask further includes a mask seal provided around the open end of the mask module. An inlet valve in the plenum module is in communication with the aerosolizing plenum. A dividing valve is provided in the closed end of the mask module between the compartment and the aerosolizing plenum. Further an exhaust valve is provided in the mask module.

These and other embodiments will be set forth in the description which follows, and in part will become apparent to those of ordinary skill in the art by reference to the following description and referenced drawings or by practice of the medication administration and training mask.

### BRIEF DESCRIPTION OF THE DRAWINGS

The accompanying drawings incorporated herein and forming a part of the specification, illustrate several aspects of the mask and together with the description serve to explain certain principles thereof. In the drawings:

FIG. 1 is an exploded perspective view of a combined medication administration and training mask.

FIG. 2 is a perspective view of the assembled mask illustrated in FIG. 1.

FIG. 3 is a side elevational view of the mask with a jet nebulizer.

FIG. 4 is a side elevational view of the mask with an ultrasonic nebulizer.

Reference will now be made in detail to the present preferred embodiment of the invention, examples of which are illustrated in the accompanying drawings.

### DETAILED DESCRIPTION

A combined medication administration and training mask 10 for an animal such as a horse or camel, comprises a body, generally designated by reference numeral 12 having a sidewall 14, a mask module 16 at a proximal end and a plenum module 18 at a distal end. The mask module 16 includes an open end 20 a compartment 22 for receiving a muzzle of an animal and a closed end 24 opposite the open end. The plenum module 18 includes a connection end 26, a closed terminal end 28 and an aerosolizing plenum 30 defined between the connection end and the terminal end.

The connection between the mask module 16 and plenum module 18 may be made by any appropriate means. In the illustrated embodiment the mask module 16 includes a recessed margin 32 and the plenum module 18 includes a cooperating lip 34 that friction fits in the recessed margin to provide a releasable connection.

A cavity 36 is provided in the terminal end 28 of the plenum module 18. The mask 10 further includes an ultrasonic nebulizer 38 and a jet nebulizer 40. A grommet seal 51 is used to seal the nebulizer 38, 40 outlet to the plenum 18. A dual purpose T piece 52 is used to connect the outlet of the jet nebulizer 38 to the plenum 18 and when turned is used as a plug. Both of the nebulizers 38, 40 are sized and shaped to be received and held in the cavity 36 so that one of the nebulizers is held in the cavity of the plenum module 18 at any one time when inserted in the cavity 36, the nebulizer 38 or 40 is substantially protected by a periphery defined by the sidewall 14 of the body 12 which has a contour that closely resembles the muzzle of the animal. Thus, the nebulizer is out of the line of sight of the animal wearing the mask 10. This can be a significant benefit during any training exercises where the animal is wearing the mask 10.

A multi-layer seal 42, made of a resilient material, is provided around the open end 20 of the mask module 16. When the mask is fitted to an animal, the muzzle sits down in the compartment 22 with the seal 42 engaging the muzzle and sealing that compartment. The seal 42 seats against the muzzle to ensure efficient function of the device by ensuring all airflow is controlled via the valves 44, 46, 48. Further, the integrity of the seal is maintained substantially leak-free despite movement and applied pressures during therapy or training.

A number of inlet valves 44 are provided in the plenum module 18 in communication with the aerosolizing plenum 30. Each inlet valve 44 allows airflow only one way: that is from the ambient environment through the valve 44 into the aerosolizing plenum 30.

A number of dividing valves 46 are provided in the closed wall or end 24 of the mask module 16 between the compartment 22 and the aerosolizing plenum 30. The dividing valves 44 are also inlet valves allowing air to flow there through in only one direction from the aerosolizing plenum 30 to the muzzle compartment 22.

A number of exhaust valves 48 are provided in the mask module 16. Exhaust valves 48 allow airflow in only one direction through the exhaust valves from the muzzle compartment 22 to the ambient environment.

During operation when an animal wearing the mask 10 inhales, ambient air is drawn through the inlet valves 44 into the aerosolizing plenum 30. Simultaneously, aerosolized medication, produced by the nebulizer 38, 40 and collected in the plenum 30, is drawn from the plenum through the dividing valves 46 into the muzzle compartment 22 and from there into the lungs of the animal. When the animal exhales, CO₂ is expelled from the lungs of the animal into the compartment 22 and through the exhaust valves 48 into the ambient environment. Simultaneously, the nebulizer 38, 40 is filling the plenum 30 with aerosolized medication to treat the animal when the animal draws its next breath. Here it should be appreciated that the nebulizer vapor is prevented from mixing with the exhaled air and being wasted. Thus, nebulization is continuous and vapor is stored in readiness for the next inhale of the animal. Consequently a lower instantaneous flow of vapor is required from the nebulizer.

As should be appreciated from viewing the drawing figures, the cavity 36 is substantially rectangular in shape. This allows an operator to conveniently and efficiently insert or remove the nebulizer 38, 40 from the cavity as desired. When in use the nebulizer 38, 40 is fully integrated into the mask 10. However, it is easily separated for cleaning. Hook and loop fasteners (not shown) may be utilized to hold the nebulizer 38, 40 in the cavity 36.

As should also be appreciated from the drawing figures, the muzzle receiving compartment 22, the aerosolizing plenum 30 and the nebulizer 38 or 40 received in the cavity 36 are all aligned along a longitudinal axis L of the animal mask 10. Significantly, the nebulizer 38, 40 is held on the terminal end 28 of the body 12 within the outer periphery defined by the sidewall 14. As such, the nebulizer 38, 40 is blocked from the view of the animal wearing the mask 10. Further, the body of the nebulizer 38, 40 is external of the aerosolizing plenum 30 to prevent contamination. Still further, it should be appreciated that the nebulizer 38, 40 sits down in the cavity 36 where it is protected and less likely to be flicked off or knocked loose from the plenum module 18 during head flicks or if the animal tries to “side swipe” the device from his muzzle. In one embodiment less than 50% of the profile of the nebulizer 38, 40 projects from the cavity 36. In another embodiment less than 40%. In yet another embodiment, less than 30%. In still another embodiment less than 20%. In yet another less than 10%. Finally, in one embodiment the entire nebulizer 38, 40 fits within the cavity 36.

When used with the mask 10, the jet nebulizer 38 facilitates rapid delivery of drugs and serums to the treatment animal. Treatment is accelerated thanks to simultaneous oxygenation of the subject. The benefits of oxygenation during treatment include reduce oxidative stress resulting from over exercise or other tissue damage. Significantly, oxidative stress triggers the release of hydrocortisone (stress hormone) which can inhibit positive immune responses. The oxygenation also helps increase the oxygen processing capacity of cell mitochondria which boosts cell activity and growth.

When used with the mask 10, the ultrasonic nebulizer 40 generates the ideal aerosol particle size for a wide range of drugs including but not limited to, saline, bronchodilators, corticosteroids and antibiotics. The reservoir and battery power pack of the ultrasonic nebulizer 40 may support two hours or more of continuous treatment.

The mask can be used with additional equipment to provide hyperoxic training. A kit for hyperoxic training includes the mask, a pulse oximeter, an oxygen supply tank, a regulating valve and an instruction manual. Hyperoxic training can be used to stress the muscular system of the training subject. More specifically, hyperoxic conditions allow longer and more frequent bouts of higher intensity training for increased strength and endurance. This is accomplished by (1) preventing a decrease in arterial oxygen concentration during high intensity exercise which causes fatigue and formation of lactic acid and (2) increasing the rate of replenishment for arterial oxygen concentration during recovery for faster dissipation of lactic acid. The SpO₂ of the subject is monitored during exercise using the pulse oximeter and controlled by changing the composition of the air the subject is breathing through the mask 10.

The mask 10 may also be used to supply the subject with oxygen therapy. Oxygen therapy offers significant benefits including: (a) managing the release of hydrocortisone which at prolonged high levels reduces the effectiveness of the immune system; and (b) increasing the oxygen holding and processing capacity of cell mitochondria (oxygen banking) which allows prolonged aerobic activity and helps prevent tissue damage.

The mask 10 may also be used to supply medical gas to reduce carbon monoxide absorption during transport in a horse float. This is significant as inhaled carbon monoxide greatly reduces respiratory efficiency by displacing oxygen in the blood. Carbon monoxide absorbed during road transport has been shown to reduce aerobic performance for days after exposure.

The mask 10 is also perfect for treadmill and hydrotherapy training as well as recovery after training or an event.

As should be appreciated, the mask 10 may be easily secured in a proper position on the animal. First, the mask 10 is manipulated so that the muzzle of the animal extends through the open end 20 of the mask module 16 and is fully received in the compartment 22. The flexible seal 42 engages around the muzzle to seal the compartment 22. A securing strap 53 is then secured over the head of the animal behind the ears so as to secure the mask 10 in place. The strap 53 may, for example, be fastened by means of a hook and loop fastener.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

What is claimed:

1. A combined medication administration and training mask for an animal, comprising:
a body having a sidewall, a mask module at a proximal end and a plenum module at a distal end;
said mask module including an open end, a compartment for receiving a muzzle of an animal and a closed end opposite said open end;
said plenum module including a connection end, a closed terminal end and an aerosolizing plenum defined between said connection end and said terminal end, said connection end of said plenum module attaching to said closed end of said mask module; and
a cavity in said terminal end of said mask module.

2. The mask of claim 1 further including a nebulizer received in said cavity.

3. The mask of claim 2, wherein said nebulizer is held in said cavity and substantially protected by a periphery defined by said sidewall of said body.

4. The mask of claim 1 further including an ultrasonic nebulizer and a jet nebulizer, said ultrasonic nebulizer and said jet nebulizer both being sized and shaped to be received and held in said cavity so that one of said ultrasonic nebulizer and said jet nebulizer is held in said cavity of said plenum module at any one time.

5. The mask of claim 1 further including a mask seal provided around said open end of said mask module.

6. The mask of claim 1, further including an inlet valve in said plenum module in communication with said aerosolizing plenum.

7. The mask of claim 6, further including a dividing valve in said closed end of said mask module between said compartment and said aerosolizing plenum.

8. The mask of claim 7, further including an exhaust valve in said mask module.

9. The mask of claim 1 can be used for hyperoxic training.

10. The mask of claim 1 can be used for hypoxic training.

11. The mask of claim 1, wherein said closed end of said mask module includes a recessed margin and said connection end of said plenum module includes a lip that friction fits in said recessed margin to provide a releasable connection.

12. The mask of claim 1, further including an attachment strap.

13. A combined medication administration and training mask for an animal, comprising:
a body having a muzzle receiving compartment and an aerosolizing plenum; and
a nebulizer connected to said body;
said muzzle receiving compartment, said aerosolizing plenum and said nebulizer all being aligned along a longitudinal axis of said animal mask.

14. The mask of claim 13 wherein said body has a sidewall defining an outer periphery, said nebulizer being held on a terminal end of said body within said outer periphery.

15. The mask of claim 13, wherein said terminal end of said body includes a cavity said nebulizer being received and held in said cavity.
